(19) United States
(12) Patent Application Publication
(10) Pub. No.: US 2026/0347301 A1
(43) Pub. Date: Nov. 5, 2026

(54) NOVEL EFFICIENT METHOD FOR PRODUCING ACRYLIC ACID AND RECOVERING ACRYLIC VALUE FROM ACRYLIC ACID OLIGOMERS

(71) Applicant: Rohm and Haas Company, Collegeville, PA (US)

(72) Inventors: Reetam Chakrabarti, Phoenixville, PA (US); Joseph F. Dewilde, King of Prussia, PA (US); James E. Elder, Friendswood, TX (US); Lan T.P. Hoang Nguyen, Doylestown, PA (US); Muhunthan Sathiosatham, Chalfont, PA (US); Mark A. Silvano, Stroudsburg, PA (US); Nelson I. Quiros, Houston, TX (US)

(21) Appl. No.: 19/391,406

(22) Filed: Nov. 17, 2025

Related U.S. Application Data
(60) Provisional application No. 63/728,510, filed on Dec. 5, 2024.

(51) Int. Cl. C07C 51/44; C07C 51/09; C07C 67/08

(52) U.S. Cl. CPC C07C 51/44; C07C 51/09; C07C 67/08

(57) ABSTRACT

A process for producing acrylic acid and recovering acrylic acid from Michael addition products comprises feeding a crude acrylic acid stream to a dehydration tower to produce an overhead stream comprising noncondensibles and lights and a bottoms stream comprising acrylic acid and Michael addition products of acrylic acid. At least a portion of the bottoms stream from the dehydration tower is fed to a finishing tower to distill the bottoms stream of the dehydration tower to produce a side draw stream comprising acrylic acid and organic carbonyl compounds, and a bottoms stream comprising acrylic acid and Michael addition products of acrylic acid. At least a portion of the side draw stream is fed to a purification unit to remove the organic carbonyl compounds and form a glacial acrylic acid stream and a bottoms stream comprising heavies. At least a portion of the bottoms stream from the finishing tower and the bottoms stream from the purification unit are reacted with a C1 to C8 alkyl alcohol in the presence of an esterification or transesterification catalyst and heating to a temperature ranging from 100 to 135 °C to form an intermediate mixture comprising a C1 to C8 alkyl acrylate and a C1 to C8 alkyl-3-hydroxy propionate and compounds of formula (VI)

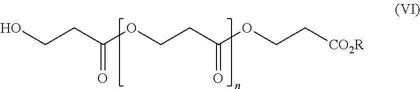

wherein R is a C1 to C8 alkyl group and n ranges from 1 to 3. The C1 to C8 alkyl-3-hydroxy propionate, the compounds of formula (VI) and any remaining Michael addition products of acrylic acid or C1 to C8 alkyl esters thereof present in the intermediate mixture are thermally cracked to form a product comprising acrylic acid and C1 to C8 alkyl acrylate.

### FIELD OF THE INVENTION

The invention relates to a method for producing acrylic acid and recovering acrylic value from acrylic acid oligomers.

### BACKGROUND OF THE INVENTION

Acrylic acid and its ester derivatives are used in the manufacture of a broad spectrum of polymer products found across multiple market segments. For example, poly-acrylic acid (PAA) formulations are commonly found in detergent applications, sodium salts of PAA are used as super absorbent polymers (SAP) found in baby diapers and other adult hygiene products. Copolymers of acrylic monomers with methacrylic monomers, styrene, butadiene and other functionalized vinyl monomers are used to manufacture dispersions and resins that are ubiquitous in consumer and industrial products such as architectural, industrial, paper and automotive coatings, performance additives for concrete admixtures and asphalts, textiles, adhesives, sealants and caulks to name just a few applications.

Commercially, acrylic monomer production is dominated by acrylic acid which serves as a platform chemical for a broad range of acrylate derivatives. Acrylic acid may be produced using a variety of different methods. For example, acrylic acid can be made via propylene oxidation, bio-based processes (e.g., using any one of glycerol, 3-hydroxypropionic acid, lactate, lactic acid, etc., as a starting material), the ethylene cyanohydrin process, the acetylene process (i.e., the Reppe process), the β-propiolactone process, acrylonitrile hydrolysis, or coupling carbon dioxide with ethylene or ethane. Industrially, most of the global capacity of acrylic acid is manufactured via the two-stage heterogeneously catalyzed oxidation of propylene over mixed metal oxide catalysts.

The crude acrylic acid (CAA) that is produced by any of the known methods contains Michael addition products, which include dimers, trimers, and higher-mers of acrylic acid. These Michael addition products may reversibly react to form acrylic acid or higher or lower oligomers.

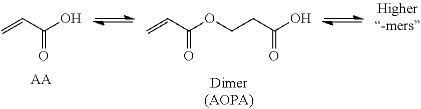

To remove Michael addition products and recover acrylic acid value from the crude acrylic acid stream, the crude acrylic acid is typically sent to a dimer cracker.

Attempts have been made to improve acrylic acid recovery in dimer crackers. Some methods utilize strong acids as a catalyst to facilitate recovery of acrylic value.

European Patent EP 1275633 discloses a process for decomposing Michael adducts of acrylic acid by reacting in the presence of an N-oxyl compound to form acrylic acid, acrylic ester, and/or alcohol.

U.S. Pat. No. 7,612,231 discloses a process for recovering acrylic acid from a mixture comprising acrylic acid dimer by carrying out decomposition of the acrylic acid dimer under reduced pressure while recovering acrylic acid by distillation in a process having a retention time at the bottom of the acrylic acid recovering device of 10 to 30 hours. U.S. Pat. No. 8,308,913 discloses an apparatus for recovering acrylic acid comprising an acrylic acid recovering device having an acrylic acid recovery device having a thin film evaporator integrated with an acrylic acid dimer pyrolysis tank.

U.S. Pat. No. 3,868,410 discloses an esterification process wherein oligomeric byproducts are converted to acrylic acid, an alkanol, or a alkylate of the alkanol by heating the oligomeric byproducts at a temperature of at least 180 °C.

The methods disclosed in the references cited above recover at most only 50 wt % of eligible acrylic acid molar equivalents.

There is a need in the art for improved methods of producing acrylic acid and recovering acrylic acid value from Michael addition products.

### SUMMARY OF THE INVENTION

The invention is directed to a process for recovering acrylic values from Michael addition products comprising:
a) feeding a stream comprising acrylic acid to a dehydration tower to produce an overhead stream comprising noncondensibles and lights and a bottoms stream comprising acrylic acid and Michael addition products of acrylic acid;
b) feeding at least a portion of the bottoms stream from the dehydration tower to a finishing tower to distill the bottoms stream of the dehydration tower to produce an acrylic acid-rich stream comprising acrylic acid and organic carbonyl compounds, and a bottoms stream comprising acrylic acid and Michael addition products of acrylic acid;
c) feeding at least a portion of the acrylic acid-rich stream to a purification unit to remove the organic carbonyl compounds and form a glacial acrylic acid stream and a bottoms stream comprising heavies;
d) optionally producing an acrylic acid ester in an esterification process from the acrylic acid-rich stream, wherein a waste stream from the esterification process comprises Michael addition products of acrylic acid; and
e) reacting at least a portion of the bottoms stream from the finishing tower, the bottoms stream from the purification unit, and/or waste stream from the esterification process with a C1 to C8 alkyl alcohol in the presence of an esterification or transesterification catalyst and heating to a temperature ranging from 80 to 135 °C to form an intermediate mixture comprising a C1 to C8 alkyl acrylate and a C1 to C8 alkyl-3-hydroxy propionate and compounds of formula (VI):

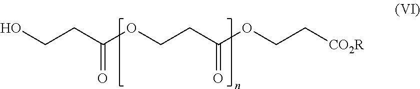

wherein R is a C1 to C8 alkyl group and n ranges from 1 to 7; and
f) thermally cracking the C1 to C8 alkyl-3-hydroxy propionate, the compounds of formula (VI) and any remaining Michael addition products of acrylic acid or C1 to C8 alkyl esters thereof present in the intermediate mixture to form a product comprising acrylic acid and C1 to C8 alkyl acrylate.

### BRIEF DESCRIPTION OF THE DRAWINGS

FIG. 1 is a diagram the reaction of a first step in the process according to an embodiment of the present invention.

FIG. 2 is a diagram of a final step in the process according to an embodiment of the present invention.

### DETAILED DESCRIPTION OF THE INVENTION

All percentage compositions are weight percentages (wt %), and all temperatures are in °C, unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system.

As used herein, the terms “Michael addition products of acrylic acid” and “dimer, trimer, and higher-mers of acrylic acid” refer to oligomers of acrylic acid. The oligomers of acrylic acid can reversibly react to form higher or lower oligomers, as well as acrylic acid.

The present invention relates to a process for producing acrylic acid and recovering acrylic value from heavy waste byproducts, such as Michael addition products of acrylic acid. As used herein “acrylic value” refers to acrylic acid and esters of acrylic acid that are useful in the production of polymers.

In the present invention, acrylic acid is produced by any known acrylic acid production process. For example, acrylic acid can be made via propylene oxidation, a bio-based process (e.g., using any one of glycerol, 3-hydroxypropionic acid, lactate, lactic acid, etc., as a starting material), an ethylene cyanohydrin process, an acetylene process (i.e., the Reppe process), a β-propiolactone process, an acrylonitrile hydrolysis process, or a process performed by coupling carbon dioxide with ethylene or ethane. The reaction mixture produced during the acrylic acid production process may be processed in one or more unit operations. These operations may include, for example, cooling processes, quenching processes, separations, purifications, etc.

Preferably, the acrylic acid is produced by an exothermic, vapor stage, two-step propylene oxidation process. In a first step, propylene is oxidized in the presence of air and a first mixed metal oxide catalyst to produce acrolein. In a second step, the acrolein is oxidized in the presence of air and a second mixed metal oxide catalyst to produce a gaseous reaction mixture comprising acrylic acid.

The gaseous reaction mixture is cooled or quenched and partially condensed to form a liquid crude acrylic acid stream. The gaseous reaction mixture typically is superheated as it comes from the two-step oxidation process, wherein it contains more heat (energy) than the amount of heat required to vaporize the condensable components of the mixture. Preferably, the cooling step removes essentially all of the amount of superheat from the condensable portion of the gaseous reaction mixture. The cooling step can be conducted directly or indirectly in one or more pieces of equipment. For example, the cooling of the gaseous reaction mixture stream can be initiated in a quench or flash vessel, or can be integrated into the bottom of the dehydration column, with or without column internals. Preferably, the cooling step is initiated or primarily conducted in the dehydration column. The cooling step advantageously is initiated by bringing the gaseous reaction mixture stream into direct contact with a liquid, which preferably has a lower temperature. In one embodiment of the invention, the gaseous reaction mixture is introduced directly into the dehydration column where it is contacted with a liquid having a temperature lower than the temperature of the gaseous reaction mixture in order to at least partially cool the gaseous reaction mixture. In a preferred embodiment of the invention, the gaseous reaction mixture is introduced into the dehydration column and contacted with a spray of a cooler liquid.

Advantageously, the gaseous reaction mixture stream is cooled to a temperature of from about 50 to about 300 °C, preferably from about 70 to about 200 °C, below the boiling point of the majority of the highest-boiling components of the mixture. Preferably, the reaction gas mixture is cooled to a temperature of from about 50 to about 200 °C, more preferably from about 60 to about 180 °C.

The at least partially cooled gaseous reaction mixture stream is dehydrated. The dehydration preferably is conducted in the dehydration column, also referred to as the dehydration tower. The dehydration tower functions to remove the majority of water from the crude acrylic acid stream exiting as the dehydration tower bottoms stream. Advantageously, the dehydration tower is operated such that there is at least a bottoms stream and an overhead stream, and in some cases a side stream. Preferably, at least a portion of the overhead stream is condensed and is returned as a reflux liquid to the dehydration tower. Preferably a portion of the bottoms stream is cooled and used to cool the gaseous reaction mixture.

Preferably, essentially all noncondensibles and lights exit the dehydration tower in the overhead stream. Examples of noncondensibles present during the production of acrylic acid include, for example, nitrogen, oxygen, CO, carbon dioxide, and unreacted hydrocarbons such as propane and propylene. Advantageously, the overhead stream is introduced into a condenser, and at least a portion of the lights and acrylic acid are condensed and returned to the dehydration tower as a reflux stream.

The dehydration tower functions, at least partially, as a distillation column. However, as noted above, the dehydration column can also serve as a contacting zone for cooling of the gaseous reaction mixture. Preferably, the temperature of the bottoms stream from the dehydration tower is less than about 120 °C. The temperature of the overhead stream from the dehydration tower is at least about 40 °C.

The vent stream from the overhead condenser on the dehydration tower may be partially recycled to the reactor system. The remaining portion of the vent stream is removed from the separation system as a purge stream.

The bottoms stream, or liquid crude acrylic acid stream, from the dehydration tower advantageously is sent to a second column, except that a portion of this stream can be employed to cool the gaseous reaction mixture. In one embodiment of the invention, a portion of the bottoms stream from the dehydration tower is sent to a heat exchanger, which can be a reboiler. However, it is noted that the process can also be operated under conditions such that the heat exchanger is a cooler, depending on whether the process design requires heating or cooling. Preferably, a portion of the bottoms stream from the dehydration tower is fed to a second column. Advantageously, the feed point is near the top of the second column. The second column preferably is a distillation column and is used in conjunction with a reboiler and a condenser.

Preferably, the overhead stream from the second column is sent to a condenser. Preferably, the condenser is operated as a “total condenser” in that essentially all of the overhead stream is condensed. However, it is possible to remove a purge stream of noncondensible compounds from this condenser. A portion of the condensate of the second column is used to provide reflux and the remaining portion of the condensate is returned to the dehydration tower for lights removal. Advantageously, the condensate from the second column condenser is used to cool the gaseous reaction mixture, either as is or after additional heat exchange.

The bottoms stream from the second column advantageously is at least partially sent to the second column reboiler. The remainder of the bottoms stream can be incinerated or can be further treated to recover acrylic values. Preferably, the majority of acrylic acid is recovered from an acrylic acid-rich stream, such as, for example, a side draw stream, from the second column. A portion of the acrylic acid will be present in the bottoms stream of the second column. For example, the acrylic acid-rich stream may comprise technical grade acrylic acid (TGAA) and the bottoms stream may comprise primarily heavies, including Michael addition products of acrylic acid, soluble polymer and polymerization inhibitor residues. Alternatively, the acrylic acid-rich stream may comprise TGAA and the bottoms stream may comprise a lower grade stream of acrylic acid, such as ester grade acrylic acid (EGAA). Preferably, the acrylic acid-rich stream comprises at least 95 wt %, more preferably at least 96 wt %, even more preferably at least 97 wt %, and still more preferably at least 98 wt % acrylic acid based on the total weight of the acrylic acid-rich stream.

The process of the invention can remove the acrylic acid-rich stream at a point above or below the point where the liquid crude acrylic acid stream is fed to the second column, depending on the purity desired, i.e. the acrylic acid-rich stream can be removed above or below the feed in the second column. The acrylic acid-rich stream may be removed as a liquid or vapor.

The temperature and pressure in the second column can be determined according to design considerations well-known to those skilled in the art. Preferably, the second column is operated pressures so that the bottoms temperature is less than 120 °C. More preferably, the second column is operated at pressures so that the bottoms temperature is less than 100 °C. The advantage of allowing the second column to operate at lower temperatures is to minimize undesired dimer, oligomer and/or polymer formation. Advantageously, the temperature of the overhead stream as it leaves the second column is from about 40 to about 90 °C when producing acrylic acid and operating the second column at a head pressure of from about 40 to about 500 mm Hg. The temperature of the bottoms stream from the second column advantageously is from about 60 to about 100 °C when producing acrylic acid.

The design details of the dehydration column and of the second column, including their operating conditions such as temperatures, pressures, flow rates, equipment sizing including column height and diameters, choice of materials of construction, arrangement and choice of type of auxiliary equipment such as heat exchangers and pumps, choice and arrangement of column internals, and location of piping including take-off streams, can readily be determined by those skilled in the art according to well-known design considerations. Examples of distillation column configurations that can be used in the process of the invention include, for example, packed columns, trayed columns, divided wall columns, multi-stage devolatilizers, and the like. Any type of tray can be employed, including bubble trays, valve trays, cross flow trays, dual flow trays, and combinations thereof for those skilled in the art. Similarly, if packing is employed, any type of packing can be used, including randomly- or regularly-spaced packing. In a preferred embodiment of the invention, the dehydration column comprises packing in its upper section, and in its lower section provides for direct cooling of the incoming gaseous reaction mixture. Surge tanks optionally can be employed within the separation system such as, for example, at one or more locations between the dehydration column and the second column.

Steel alloys such as 316 SS and other higher alloys may be used as the material(s) of construction for the process equipment by using criteria well known to those skilled in the art for process equipment to be used in the distillation of corrosive streams. Alternatively, the process equipment can be partially constructed using copper or copper-containing alloys, such as various alloys sold under the name Monel. While not wishing to be bound by any theory, it is the believed that the presence of copper inhibits the undesired polymerization of acrylic acid.

The use of inhibitors is preferred in the process of the invention, regardless of the choice of materials of construction. Various compounds are well known to inhibit the reaction of acrylic acid, and are commercially available. Examples of preferred inhibitors include complexes and salts of manganese compounds, copper complexes and salts thereof, hydroquinone (HQ), monomethyl ether hydroquinone (MEHQ), phenothiazine (PTZ), 2,2,6,6-tetramethyl-1-piperidinyloxy (TEMPO), its derivatives, and related compounds such as 4-hydroxy TEMPO. Combinations of inhibitors can be employed that offer synergistic interactions and provide more effective polymerization inhibition than the additive performance of the individual inhibitors. In a preferred embodiment of the invention, a mixture of a source of soluble manganese ions, such as manganese acetate, and 4-hydroxy TEMPO are employed in the dehydration column as an inhibitor. Hydroquinone can also be added to this inhibitor mixture. It is also preferred, as is well-known in the art, to employ a molecular oxygen or air to the second column, as oxygen is known to be a polymerization retarder. The inhibitor is employed in an amount sufficient to prevent or reduce the polymerization of acrylic acid, as is well known to those skilled in the art.

Preferably, the dehydration tower and second column form a coupled distillation tower system. The coupled distillation tower system comprises the dehydration tower and a finishing tower (i.e., the second column). Other suitable process configurations are possible and are known to those skilled in the art which use intermediate tanks to contain certain process streams prior to being fed to each tower.

Preferably, at least a portion of the acrylic acid in the acrylic acid-rich stream is fed to an esterification process to convert acrylic acid to an ester of acrylic acid. Preferably, the acrylic acid ester formed in the esterification process is selected from the group consisting of methyl acrylate, ethyl acrylate, butyl acrylate, and 2-ethyl hexyl acrylate. More preferably, the acrylic acid ester formed in the esterification process is butyl acrylate. The esterification process may comprise one or more unit operations to purify the acrylic acid ester to produce a product stream and a waste stream comprising heavies including Michael addition products of acrylic acid.

The acrylic acid in the acrylic acid-rich stream of the second column comprises residual organic carbonyl compounds that are byproducts of the heterogeneously-catalyzed oxidation of propylene, such as maleic acid, maleic anhydride, propionic acid, acetic acid, formaldehyde, furfuraldehyde, benzaldehyde, acrolein, protoanemonin, acrylic acid dimer among others. High levels of maleic anhydride, aldehydes and other similar species, can render the acrylic acid unsuitable for use in certain products, such as the formation of super absorbent polymers (SAP), which require low aldehyde concentrations. Examples of the residual organic carbonyl compounds include furfural, benzaldehyde, acrolein, maleic acid, and formaldehyde. The amount of these compounds varies according to reactor catalyst performance and may be present individually in the acrylic acid-rich stream in amounts ranging from 10 ppm to 2000 ppm depending on the individual component.

To further purify the acrylic acid of the acrylic acid-rich stream of the second column and reduce the amount of residual organic carbonyl compounds, and preferably to reduce the amount of residual aldehydes, at least a portion of the acrylic acid-rich stream is fed to a purification unit to remove at least a portion residual organic carbonyl compounds and to produce a glacial acrylic acid (GAA) stream. Preferably, the acrylic acid-rich stream of the second column is fed to a surge tank before proceeding to the purification unit. The surge tank may help manage fluctuations in flow rates or pressure, and may also be used as to divert the acrylic acid-rich stream into one or more streams. For example, a first portion of the acrylic acid-rich stream may be fed to purification unit, and a second portion of the acrylic acid-rich stream may be fed to a downstream unit, such as an ester unit for manufacturing acrylic acid esters from the side draw stream. Preferably, at least a portion of the acrylic acid-rich stream is fed to the purification unit and a remainder of the side draw stream is fed to an ester unit for manufacturing acrylic acid esters.

In one embodiment, the purification unit comprises a reactive distillation tower to form a glacial acrylic acid (GAA) stream. A chemical additive is fed to the reactive distillation tower to react with the residual organic carbonyl compounds and form stable carbonyl adducts that are separable from the acrylic acid by distillation. The chemical additive may be added to the tower itself, or to the feed stream to the tower. The additive can be added directly to said feed or via a device that will efficiently mix the additive with the acrylic acid stream so as to maximize the formation of the adducts of the additive with the unwanted carbonyl compounds with the lowest practical amount of chemical additive compound added. These heavy carbonyl adducts are then partitioned into the bottoms of the reactive distillation column. GAA is removed from the reactive distillation column as a distillate. As some chemical additives may generate water as a byproduct of their reaction with said residual carbonyl compounds and water being a light component relative to acrylic acid, GAA with a lower water content may alternately be drawn from the reactive distillation tower as a side draw stream. The GAA side draw stream may be removed and subjected to cooling through a heat exchanger prior to collection and storage.

The chemical additive may comprise compounds such as amines including ethanolamine, ethylenediamine, hydrazine, hydrazine hydrate and its derivatives, aminoguanidine and its derivatives, mono-phenyl-amines such as aniline and its analogs which may have a plurality of substituents on the ortho, meta and para positions of the phenyl ring such as methyl, methoxy, ethyl, ethoxy and other substituents, phenylenediamine isomers such as ortho, para and meta-phenylenediamines and their derivatives, and other similar compounds that will react with the carbonyl group of the compound converting it to a stable, heavy compound separable from acrylic acid by distillation. The additive compound may be used individually or in mixtures or combination thereof with other additives.

In an alternative embodiment, the purification unit comprises a crystallizer to form a glacial acrylic acid (GAA) stream. The crystallizer may use any known crystallization methods, such as, for example, static, falling film, suspension, or others, as well as combinations thereof. Preferably, the crystallizer comprises a suspension crystallizer. The crystallizer removes residual organic carbonyl compounds from the acrylic acid-rich stream. In addition to the acrylic acid product, the crystallizer also forms a mother liquor. The mother liquor may comprise acrylic acid, dimers, trimers, and higher oligomers, as well as a relatively high level of polymerization inhibitors. Depending on the crystallization technology, the mother liquor may be subjected to additional crystallization cycles to increase the yield of GAA and/or reduce the amount of residual carbonyl or aldehydes, or it may be recycled back to the finishing tower as supplementary recycle inhibitor or may be disposed of by incineration.

The GAA produced in the purification unit preferably comprises less than 20 ppm of furfural, benzaldehyde, formaldehyde, and acrolein combined, i.e., the total content of furfural, benzaldehyde, formaldehyde, and acrolein in the GAA is less than 20 ppm. More preferably, the GAA comprises less than 10 ppm of furfural, benzaldehyde, formaldehyde, and acrolein combined, and still more preferably, less than 5 ppm of furfural, benzaldehyde, formaldehyde, and acrolein combined.

In at least one preferred embodiment, the GAA produced in the purification unit comprises less than 5 ppm of each of furfural, benzaldehyde, formaldehyde, and acrolein. More preferably, the GAA comprises less than 3 ppm of each of furfural, benzaldehyde, formaldehyde, and acrolein, and even more preferably less than 2 ppm of each of furfural, benzaldehyde, formaldehyde, and acrolein.

The bottoms stream of the second column, the bottoms of the reactive distillation column or the mother liquor from the crystallizer, and/or the waste stream from the esterification process are combined to form a heavies stream. The heavies stream comprises acrylic acid, the dimer of acrylic acid (3-(acryloyloxy) propanoic acid or AOPA), acrylic acid trimer, and acrylic acid tetramer. Higher oligomers of acrylic acid may also be present. The dimer is preferably the majority component of the Michael addition products. More preferably, the dimer is the majority component in the heavies stream.

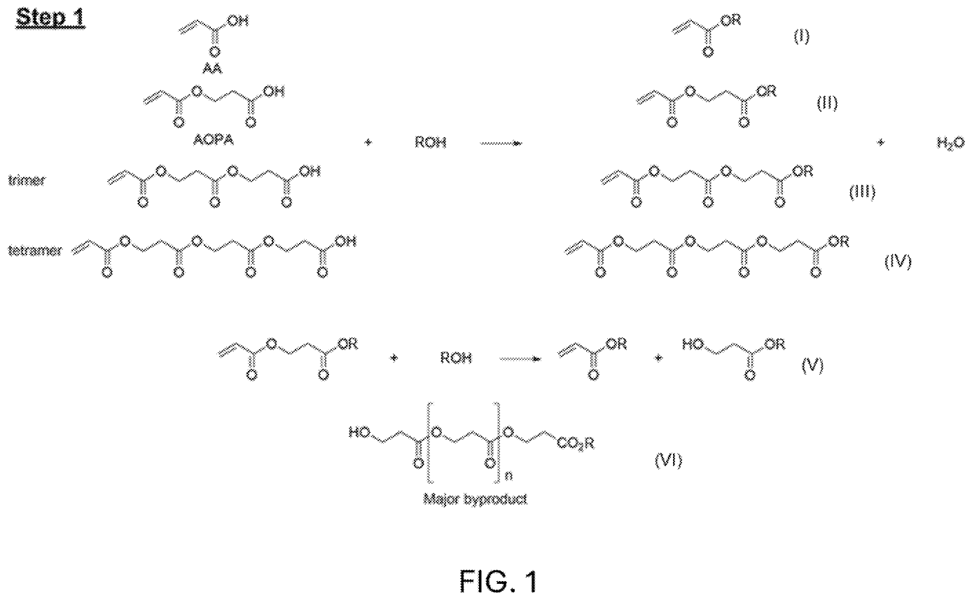

In the inventive process, acrylic value recovery is performed by reacting the heavies stream with a C1 to C8 alkyl alcohol (i.e., ROH, where R is a C1 to C8 alkyl group) to form an intermediate mixture as shown in FIG. 1.

When reacted with a C1 to C8 alkyl alcohol, C1 to C8 alkyl esters of the acrylic acid (i.e., C1 to C8 alkyl acrylates) and its Michael addition products (compounds of formulae (I) to (IV) in FIG. 1) are formed.

The alkoxy-3-acryloyloxypropanoic acid of formula (II) further reacts with the C1 to C8 alkyl alcohol to form an alkyl-3-hydroxy propionate of formula (V) and, as a major byproduct, oligomers thereof (compounds of formula (VI)).

Thus, the first step of the recovery process yields an intermediate stream mixture comprising compounds of formulae (I) to (VI).

The alkyl-3-hydroxy propionate of formula (V) and its oligomers (compounds of formula (VI)) are generally regarded as an undesirable byproducts. However, the present inventors have discovered that acrylic value recovery can be improved by driving the reaction to convert the Michael addition products to form compounds of formulae (V) and (VI) and then thermally cracking to recover acrylic value. Significantly higher acrylic value recoveries can be achieved compared to traditional dimer cracker processes.

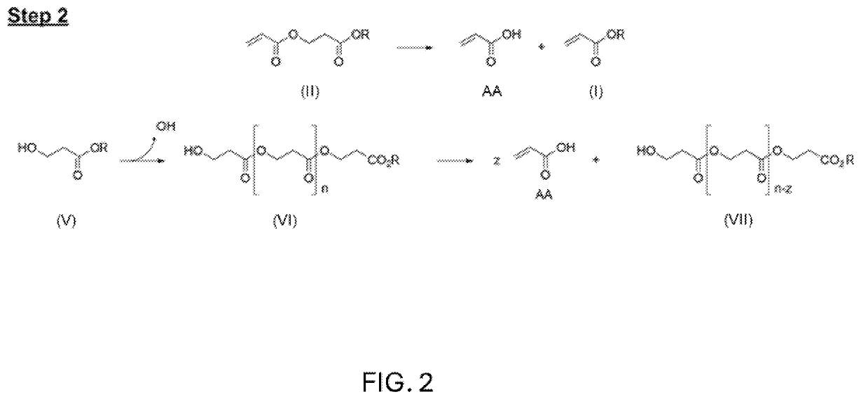

In a subsequent step (FIG. 2), the intermediate reaction mixture is thermally cracked to form acrylic acid and C1 to C8 alkyl acrylates. In the thermal cracking reaction, alkoxy-3-acryloyloxypropanoic acid of formula (II) forms acrylic acid and a C1 to C8 alkyl acrylate. The higher oligomeric compounds of formulae (III) and (IV) are thermally cracked to form acrylic acid and lower oligomers, which are further reduced to form acrylic acid and the C1 to C8 alkyl acrylate. The compounds of formulae (V) and (VI) are thermally cracked to form acrylic acid and lower oligomers of formula (VI), which further react to form acrylic acid and the corresponding C1 to C8 alkyl acrylate.

The C1 to C8 alkyl alcohol preferably comprises methanol, ethanol, butanol, and 2-ethyl hexyl alcohol. More preferably, the C1 to C8 alkyl alcohol is butanol.

When the C1 to C8 alkyl alcohol is butanol, the first step of the recovery process forms an intermediate mixture comprising butyl acrylate (compound (I) where R is a butyl group), butyoxy-3-acryloyloxypropanoic acid (BAOPA; compound (II) where R is a butyl group), and higher oligomers (compounds (III) and (IV) where R is a butyl group).

BAOPA (compound (II) where R is a butyl group) further reacts with butanol to form butyl acrylate and butyl-3-hydroxy propionate (BBHP; compound (V) where R is a butyl group). Higher oligomers of BBHP (compounds of formula (VI)) are also formed as a major byproduct.

In a subsequent step, the intermediate mixture comprising compounds (II) to (VI) where R is a butyl group is thermally cracked. The BAOPA is reduced to acrylic acid and butyl acrylate. The BBHP and higher oligomers thereof form acrylic acid and lower oligomers of BBHP, which further react to recover additional acrylic value.

The first step of the recovery process is conducted in the presence of an esterification or transesterification catalyst. Esterification and transesterification catalysts include, for example, ion exchange resins and strong acids, such as hydrochloric acid, sulfuric acid, p-toluenesulfonic acid, phosphoric acid, and methanesulfonic acid. Preferably, the catalyst is a strong acid, more preferably sulfuric acid.

A polymerization inhibitor is added during the first step of the recovery process, the thermal cracking step, or both. Alternatively, a polymerization inhibitor may already be present in the heavies stream fed to the recovery process.

The first step of the recovery process is conducted at a temperature ranging from 80 to 135 °C, preferably from 90 to 135 °C, more preferably from 100 to 135 °C, and still more preferably from 110 to 135 °C.

The thermal cracking step is preferably conducted at a temperature of at least 135 °C, more preferably 150 °C, and even more preferably at least 175 °C.

Acrylic values may be removed at any step of the recovery process. For example, acrylic acid and/or the C1 to C8 alkyl acrylates may be removed as a vapor stream or distillate from the reaction of the first step of the recovery process.

A separation step may be conducted between the first step of the recovery process and the thermal cracking step. For example, the intermediate stream may be subjected to distillation to recover additional acrylic values before the thermal cracking step. If present, the distillation step is preferably carried out at a temperature ranging from 40 to 80 °C, preferably from 45 to 70 °C, and at an absolute pressure less than 70 mmHg, more preferably less than 40 mmHg, and even more preferably less than 10 mmHg, and most preferably approaching 0 mmHg.

The present invention surprisingly enables the recovery of a significant amount of the Michael addition products of acrylic acid from the feed. For example, when butanol is used as the C1 to C8 alkyl alcohol, approximately 85 to 90 wt % of the Michael addition products of acrylic acid are recovered as acrylic acid or butyl acrylate. This represents a significant improvement over conventional dimer cracking methods.

### EXAMPLES

### Example 1

### Step 1—Esterification

A 1000 ml 4-neck round-bottom flask equipped with a magnetic stirrer, thermocouple, and a Dean-Stark distillation head was charged with AOPA (250 g; Aldrich containing 19.4% acrylic acid, 32.9% AOPA, 21.4% trimer, and 21.4% tetramer), BuOH (300 g, 4.05 mol), sulfuric acid (15 g, 0.15 mol), and PTZ (1 g). The resulting solution was mixed well and a sample was taken for NMR analysis. The contents of the flask were slowly heated to 135 °C and the distillate was collected in the Dean-Stark apparatus. The distillation commenced when the pot temperature was at 105 °C and the vapor temperature was between 95 °C and 100 °C. The total amount of water collected was 34 g and the organic distillate (D1) weighed 18 g and was analyzed by quantitative NMR and gas chromatography (GC). The heating was turned off when the distillation ceased and the pot contents were allowed to cool to room temperature. The soluble polymers in the reaction mixture were filtered off using a Buchner funnel and weighed 8 g upon air drying. Filtrate weighed 498 g.

### Step 2—Distillation

The filtrate from the above step was transferred back to the round-bottom flask and the Dean-Stark head was replaced with a short-path distillation head and the pressure was reduced to <1 mmHg absolute. The contents of the flask were heated to 60 °C and the distillate was collected at the vapor temperature range of 33 °C to 38 °C This distillation was done to remove excess BuOH and product BA before the thermal cracking step. The heating turned off and the vacuum was released when the vapor temperature dropped to below 25 °C The distillate (D2) collected weighed 247 g and was analyzed by quantitative NMR and GC.

### Step 3—Thermal Cracking

The setpoint was increased to 180 °C and the pressure was reduced to 100 mmHg. The distillate began to collect when the pot temperature was at 135 °C and continue to collect for 60 minutes at the vapor temperature range of 100 °C to 135 °C After 60 minutes distillate collection ceased and the amount of distillate (D3) collected was 242 g. Pot residue, black solid, weighed 7.5 g.

Heavies recovery was 87%. The results for Experiment 1 are shown below in Table 1.

TABLE 1

| | molar | mol % |
|---|---|---|
| Overall C3 yield (AA + BA) | 1.95 | 90% |
| Heavies recovery | 0.37 | 87% |
| Overall C4 yield (BA + BuOH) | 2.73 | 89% |

### Example 2

### Step 1—Esterification

A 1000 ml 4-neck round-bottom flask equipped with a magnetic stirrer, thermocouple, and a Dean-Stark distillation head was charged with the bottoms from a glacial acrylic acid (GAA) production process (250 g), BuOH (300 g, 4.05 mol), sulfuric acid (15 g, 0.15 mol), and PTZ (1 g). The bottoms of the GAA production process contained 45.7% acrylic acid, 33.4% AOPA, and 8.0% trimer. The resulting solution was mixed well and a sample was taken for NMR analysis. The contents of the flask were slowly heated to 135 °C and the distillate was collected in the Dean-Stark apparatus. The distillation commenced when the pot temperature was at 107.5 °C and the vapor temperature was between 98 °C and 110 °C. The total amount of water collected was 45 g and the organic distillate (D1) weighed 23 g and was analyzed by quantitative NMR and GC.

### Step 2—Distillation

The product mix from the above step was subjected to distillation. The Dean-Stark head was replaced with a short-path distillation head and the pressure was reduced to <1 mmHg absolute. The contents of the flask were heated to 60 °C and the distillate was collected at the vapor temperature range of 30 °C to 35 °C This distillation was done to remove excess BuOH and product BA before the thermal cracking step. The heating turned off and the vacuum was released when the vapor temperature dropped to below 25 °C The distillate (D2) collected weighed 267 g and was analyzed by quantitative NMR and GC.

### Step 3—Thermal Cracking

The setpoint was increased to 180 °C and the pressure was reduced to 200 mmHg. The distillate began to collect when the pot temperature was at 145 °C and continue to collect for 40 minutes at the vapor temperature range of 85 °C to 135 °C. After 60 minutes distillate collection ceased and the amount of distillate (D3) collected was 131 g. Pot residue, black solid, weighed 55 g.

Heavies recovery was 74%. The results of Example 2 are shown below in Table 2.

TABLE 2

| | molar | mol % |
|---|---|---|
| Overall C3 yield (AA + BA) | 2.64 | 88% |
| Heavies recovery | 1.05 | 74% |
| Overall C4 yield (BA + BuOH) | 3.13 | 89% |

### Example 3

### Step 1—Esterification

A 1000 ml 4-neck round-bottom flask equipped with a magnetic stirrer, thermocouple, and a Dean-Stark distillation head was charged with a GAA bottoms stream (250 g), BuOH (300 g, 4.05 mol), sulfuric acid (15 g, 0.15 mol), and PTZ (1 g). The GAA bottoms stream contained 45.7% acrylic acid, 33.4% AOPA, and 8.0% trimer. The resulting solution was mixed well and a sample was taken for NMR analysis. The contents of the flask were slowly heated to 135 °C and the distillate was collected in the Dean-Stark apparatus. The distillation commenced when the pot temperature was at 107.5 °C and the vapor temperature was between 98 °C and 110 °C. The total amount of water collected was 46 g and the organic distillate (D1) weighed 10 g and was analyzed by quantitative NMR and GC.

### Step 2—Distillation

The product mix from the above step was subjected to distillation. The Dean-Stark head was replaced with a short-path distillation head and the pressure was reduced to 70 mmHg. The contents of the flask were heated to 115 °C and the distillate was collected at the vapor temperature range of 60 °C to 80 °C. This distillation was done to remove excess BuOH and product BA before the thermal cracking step. The heating turned off and the vacuum was released when the vapor temperature dropped to below 25 °C. The distillate (D2) collected weighed 270 g and was analyzed by quantitative NMR and GC.

### Step 3—Thermal Cracking

The setpoint was increased to 180 °C and the pressure was reduced to 200 mmHg. The distillate began to collect when the pot temperature was at 145 °C and continue to collect for 40 minutes at the vapor temperature range of 85 °C to 125 °C. After 60 minutes distillate collection ceased and the amount of distillate (D3) collected was 110 g. Pot residue, black solid, weighed 85 g.

Heavies recovery was 55%. The results of Example 3 are shown below in Table 3. The reduced heavies recovery was believed to be the result of the higher temperature and pressure in the distillation step.

TABLE 3

| | molar | mol % |
|---|---|---|
| Overall C3 yield (AA + BA) | 2.37 | 78% |
| Heavies recovery | 0.79 | 55% |
| Overall C4 yield (BA + BuOH) | 2.97 | 85% |

### Example 4

### Step 1—Esterification

A 1000 ml 4-neck round-bottom flask equipped with a magnetic stirrer, thermocouple, and a Dean-Stark distillation head was charged with GAA bottoms stream (250 g), BuOH (300 g, 4.05 mol), sulfuric acid (22.5 g, 0.23 mol), and PTZ (1 g). The GAA bottoms stream contained 45.7% acrylic acid, 33.4% AOPA, and 8.0% trimer. The resulting solution was mixed well and a sample was taken for NMR analysis. The contents of the flask were slowly heated to 135 °C and the distillate was collected in the Dean-Stark apparatus. The distillation commenced when the pot temperature was at 107.5 °C and the vapor temperature was between 98 °C and 115 °C. The total amount of water collected was 46 g and the organic distillate (D1) weighed 9.5 g and was analyzed by quantitative NMR and GC.

### Step 2—Distillation

The product mix from the above step was subjected to distillation. The Dean-Stark head was replaced with a short-path distillation head and the pressure was reduced to <1 mmHg absolute. The contents of the flask were heated to 60 °C and the distillate was collected at the vapor temperature range of 35 °C to 45 °C. This distillation was done to remove excess BuOH and product BA before the thermal cracking step. The heating turned off and the vacuum was released when the vapor temperature dropped to below 25 °C. The distillate (D2) collected weighed 270 g and was analyzed by quantitative NMR and GC.

### Step 3—Thermal Cracking

The setpoint was increased to 180 °C and the pressure was reduced to 200 mmHg. The distillate began to collect when the pot temperature was at 145 °C and continue to collect for 40 minutes at the vapor temperature range of 85 °C to 125 °C. After 60 minutes distillate collection ceased and the amount of distillate (D3) collected was 155 g. Pot residue, black solid, weighed 85 g.

Heavies recovery was 90%. The results from Example 4 are shown below in Table 4.

TABLE 4

| | molar | mol % |
|---|---|---|
| Overall C3 yield (AA + BA) | 2.87 | 95% |
| Heavies recovery | 1.29 | 90% |
| Overall C4 yield (BA + BuOH) | 3.13 | 89% |

1. A process for recovering acrylic values from Michael addition products comprising:
a) feeding a stream comprising acrylic acid to a dehydration tower to produce an overhead stream comprising noncondensibles and lights and a bottoms stream comprising acrylic acid and Michael addition products of acrylic acid;
b) feeding at least a portion of the bottoms stream from the dehydration tower to a finishing tower to distill the bottoms stream of the dehydration tower to produce an acrylic acid-rich stream comprising acrylic acid and organic carbonyl compounds, and a bottoms stream comprising acrylic acid and Michael addition products of acrylic acid;
c) feeding at least a portion of the acrylic acid-rich stream to a purification unit to remove the organic carbonyl compounds and form a glacial acrylic acid stream and a bottoms stream comprising heavies;
d) optionally producing an acrylic acid ester in an esterification process from the acrylic acid-rich stream, wherein a waste stream from the esterification process comprises Michael addition products of acrylic acid; and
e) reacting at least a portion of the bottoms stream from the finishing tower, the bottoms stream from the purification unit, and/or waste stream from the esterification process with a C1 to C8 alkyl alcohol in the presence of an esterification or transesterification catalyst and heating to a temperature ranging from 80 to 135 °C to form an intermediate mixture comprising a C1 to C8 alkyl acrylate and a C1 to C8 alkyl-3-hydroxy propionate and compounds of formula (VI): 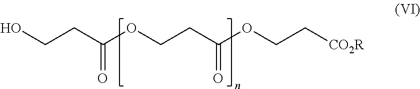
wherein R is a C1 to C8 alkyl group and n ranges from 1 to 7; and
f) thermally cracking the C1 to C8 alkyl-3-hydroxy propionate, the compounds of formula (VI) and any remaining Michael addition products of acrylic acid or C1 to C8 alkyl esters thereof present in the intermediate mixture to form a product comprising acrylic acid and C1 to C8 alkyl acrylate.

2. The process of claim 1, wherein step e) comprises reacting at least a portion of the waste stream from the esterification process

3. The process of claim 1, wherein the C1 to C8 alkyl alcohol is selected form the group consisting of methanol, ethanol, butanol, and 2-ethyl hexyl alcohol.

4. The process of claim 3, wherein the C1 to C8 alkyl alcohol is butanol.

5. The process of claim 1, further comprising removing C1 to C8 alkyl acrylate and/or C1 to C8 alkyl alcohol from the intermediate mixture before the thermal cracking step.

6. The process of claim 5, wherein the C1 to C8 alkyl acrylate and/or C1 to C8 alkyl alcohol are removed from the intermediate mixture by distillation.

7. The process of claim 1, wherein thermally cracking the C1 to C8 alkyl-3-hydroxy propionate and the compounds of formula (VI) comprises heating the C1 to C8 alkyl-3-hydroxy propionate and the compounds of formula (VI) to a temperature at least 135 °C.

8. The process of claim 7, wherein thermally cracking the C1 to C8 alkyl-3-hydroxy propionate, the compounds of formula (VI) and any remaining Michael addition products of acrylic acid or C1 to C8 alkyl esters thereof present in the intermediate mixture comprises heating the C1 to C8 alkyl-3-hydroxy propionate and the compounds of formula (VI) to a temperature at least 150 °C.

9. The process of claim 8, wherein thermally cracking the C1 to C8 alkyl-3-hydroxy propionate and the compounds of formula (VI) comprises heating the C1 to C8 alkyl-3-hydroxy propionate and the compounds of formula (VI) to a temperature at least 175 °C.

10. The process of claim 1, wherein the purification unit comprises a crystallizer.

11. The process of claim 1, wherein the purification unit comprises a reactive distillation column and a chemical additive is fed to the reactive distillation column to react with the organic carbonyl compounds in the side draw stream to form heavy carbonyl adducts that are reacted with the C1 to C8 alkyl alcohol.

12. The process of claim 1, wherein the acrylic acid in the stream fed to the dehydration tower is produced by a method selected from the group consisting of propylene oxidation-based process, a bio-based process, an ethylene cyanohydrin-based process, an acetylene-based process, a β-propiolactone-based process, an acrylonitrile hydrolysis-based process, or a process performed by coupling carbon dioxide with ethylene or ethane.

13. The process of claim 12, wherein the acrylic acid in the stream fed to the dehydration tower is produced by an exothermic vapor phase propylene oxidation-based process.
